(19) United States
(12) Patent Application Publication
(10) Pub. No.: US 2025/0345312 A1
(43) Pub. Date: Nov. 13, 2025

(54) FORMULATION AND METHOD FOR TREATMENT OF ALZHEIMER'S DISEASE, STROKE AND DIABETIC RETINOPATHY

(71) Applicant: AMRITA VISHWA VIDYAPEETHAM, Kochi (IN)

(72) Inventors: Krishnakumar Menon, Kochi (IN); Gopi Mohan Chethampadi, Kochi (IN); Sunitha Subhramanian, Kochi (IN)

(21) Appl. No.: 19/217,100

(22) Filed: May 23, 2025

Related U.S. Application Data
(63) Continuation of application No. PCT/IN2023/051090, filed on Nov. 24, 2023.

(30) Foreign Application Priority Data
Nov. 24, 2022 (IN) 202241067658

(51) Int. Cl. A61K 31/416; A61K 9/00; A61K 9/08; A61K 47/10; A61K 47/14; A61P 25/28

(52) U.S. Cl. CPC A61K 31/416; A61K 9/0019; A61K 9/0053; A61K 9/08; A61K 47/10; A61K 47/14; A61P 25/28

(57) ABSTRACT

The invention discloses formulation for use in the treatment of diseases such as Alzheimer's Disease, Stroke, and Diabetic Retinopathy as well as a method for treating said diseases by administering said formulation. The formulation comprises a therapeutically effective amount of (1-H indazole-4yl-)methanol, ethanol, and Miglyol812N at a ratio of 1 drug, to 10 Ethanol to 90 Miglyol. The formulation helps in improving retention, bioavailability, and blood-brain barrier penetration of (1-H indazole-4yl-)methanol and was effective in suppressing inflammation by blocking hGMF-β phosphorylation and activity.

### CROSS REFERENCE TO RELATED APPLICATIONS

This application is a continuation application of International Patent Application No. PCT/IN2023/051090, filed on Nov. 24, 2023, which claims priority from Indian Patent Application No. 202241067658, filed on Nov. 24, 2022, the contents of each of which are incorporated by reference herein in their entireties.

### FIELD OF THE INVENTION

The present invention pertains to the field of pharmaceutical sciences. Specifically, the invention discloses a formulation comprising a compound of Formula (I), or its pharmaceutically acceptable salt along with carrier combinations that facilitate improved retention, bioavailability, and penetration of the blood-brain barrier (BBB). The present invention also discloses a method of preparing said formulation as well as a method of treating Alzheimer's, Stroke, and Diabetic Retinopathy by administering a therapeutically effective amount of said formulation.

### BACKGROUND

Alzheimer's disease (AD) is an irreversible, progressive brain disease that causes problems with memory, thinking, and behavior. AD is the most common type of dementia, a general term for loss of memory and other mental abilities. Brains with AD show the build-up of a sticky plaque made of a protein called beta-amyloid that induces memory loss. When afflicted with AD, the immune system, which typically rids the body of toxic substances, becomes imbalanced and inefficient in clearing those plaques. In fact, the immune response at the sites of damage is thought to aggravate the situation, and the current concept of AD including diabetic retinopathy (DR) pathology is immensely aggravated by the immune response. Increasing evidence indicates that Amyloid β has the ability to bind to microbial cell wall and has been shown to entrap microbes creating inflammatory response. Further evidence indicates that due to “leaky gut” microbial entry from the gut contributes to the significant involvement of gut-brain axis in neurodegenerative diseases such as AD and Multiple Sclerosis (MS) including DR wherein, a gut-eye axis exists. Glia maturation factor-β is instrumental in augmenting this immune response.

Stroke is a medical condition in which poor blood flow to the brain causes cell death. There are two main types of stroke: ischemic, due to lack of blood flow, and hemorrhagic, due to bleeding. Both cause parts of the brain to stop functioning properly.

DR is a diabetes complication that affects the eyes. It is caused by damage to the blood vessels of the light-sensitive tissue at the back of the eye (retina). At first, DR might cause no symptoms or only mild vision problems. But it can lead to blindness. The condition can develop in anyone who has type 1 or type 2 diabetes.

Immune response in AD or Stroke or DR has significant influence in enhancing the neurodegenerative process and the clinical outcome. These three debilitating diseases together affect millions of people worldwide. Current therapies in AD or Stroke or DR modulate disease differently as they do not target the immune arm of these diseases. Treatment directed towards combating the immune arm of these diseases is still not being practiced.

Recent findings show that Glia Maturation Factor-beta (GMF-β), a 141 amino acid protein predominantly expressed in the brain is upregulated in AD/stroke/DR and propels the inflammatory response making diseases difficult to treat. GMF-β belongs to the actin-binding proteins (ADF) structural family. GMF-β appears to play a role in the differentiation, maintenance, and regeneration of the nervous system. Phosphorylation of GMF-β on amino acid residues Thr27, Ser53, Ser72 and Ser83 by different protein kinases is critical in its activation and regulation of inflammatory response. Therefore, one of the ways by which the function of GMF-β can be regulated is by phosphorylation inhibition. However, developing inhibitors against these generic protein kinases involved in the phosphorylation of human GMF-β is highly non-specific which could lead to adverse drug reactions.

U.S. Pat. Nos. 8,263,597B2, 11,110,078B2 disclose indazole based compound as CCR1 antagonist which is described to be useful against autoimmune diseases such as Rheumatoid Arthritis and MS. US20100292231A1 describes indazole scaffolds for inflammatory disorders, demyelinating disorders, FLT3-mediated disorders, CSF-1R-mediated disorders, cancers and leukemias. WO2010065776 describes methods for identifying new therapeutic agents using human cell-based models. U.S. Pat. No. 9,725,450B2 discloses certain amino-substituted purine compounds, compositions, and methods for treating or preventing certain diseases by modulating the expression of various genes. US20050009876A1 describes a method for treating or preventing diseases associated with protein kinases using indazole based scaffold compounds. A method of treating a kinase-dependent condition, especially inflammation or cancer, by administering anilino-pyrimidine compounds is described in US20070244140 A1. The US patent publication US20110136148A1 relates to methods and kits for the immunodetection of cells or samples that express soluble or secreted GMF-β antigens. The use of the antibody in the treatment and detection of cancer, AD, and dementia is described. However, the antibody is directed to the soluble form, and does not cross the BBB and its effectiveness in the treatment of autoimmune diseases such as MS is unclear. U.S. Pat. No. 11,110,078B2 discloses the composition and method for treatment of diseases relating to CNS inflammation useful in multiple sclerosis (MS), AD, Parkinson's disease (PD) or immunomodulation leading to effective immunotherapy in Cancer. However, a major drawback of the disclosure shown in U.S. Pat. No. 11,110,078B2 patent is Indazole-4-yl-methanol (GMFBI.1) stand-alone inability to cross the intact BBB and has high clearance leading to poor bioavailability when given in saline. Similarly, none of these documents describes a specific formulation comprising a compound for targeting the phosphorylation of brain-specific protein GMF-β which is associated with proinflammatory response in the brain. The present invention describes a formulation that overcomes the above-specified disadvantages, wherein the bioavailability of the drug is increased and is effective in the treatment of diseases, especially AD, Stroke, DR, and MS. The significance of this formulation lies in the fact that the presence of a combination of GMFBI.1-Ethanol-Miglyol812N at a ratio of 1 drug, to 10 ethanol to 90 miglyol812N (for example 1 mg of GMFBI.1 drug:10 microliter of ethanol: 90 microliter of miglyol 812N) helps the drug to cross the BBB and that absence of miglyol has no effect of the drug due to non-availability of GMFBI.1 inhibitor in the brain as shown in FIGS. 3 and 4. The present invention also discloses methods of treatment of diseases such as AD, Stroke, and DR using said formulation.

### Objectives of the Invention

To provide a formulation of (1-HIndazol-4-yl)-methanol (GMFBI.1) represented by compound of formula (I) which enables efficient and increased delivery of GMFBI.1 across the BBB and to increase the retention of GMFBI.1 in circulation to increase its bioavailability. To provide a method for the preparation of said formulation. To provide a method of treatment of AD, Stroke and DR by administering a therapeutically effective amount of said formulation.

### SUMMARY OF THE INVENTION

The present invention discloses a formulation comprising GMFBI.1 represented by a compound of formula (I) along with ethanol and miglyol812N at a ratio of 1 drug, to 10 Ethanol to 90 Miglyol (For example, 1 mg of GMFBI.1 drug:10 microliter of ethanol: 90 microliter of miglyol812N). The present invention also discloses a method of preparation of said formulation and the use of said formulation in the treatment of various diseases such as AD, Stroke, and DR.

Another aspect of the invention pertains to a method of treatment of AD comprising administration of the formulation comprising a therapeutically effective amount of GMFBI.1 represented by a compound of formula (I), ethanol and miglyol812N at a ratio of 1 drug, to 10 Ethanol to 90 miglyol (For example, 1 mg of GMFBI.1 drug:10 microliter of ethanol: 90 microliter of miglyol 812N).

Yet another aspect of the invention pertains to a method of treatment of Stroke comprising administration of the formulation comprising a therapeutically effective amount of GMFBI.1 represented by compound of formula (I), ethanol, and miglyol812N at a ratio of 1 drug, to 10 Ethanol to 90 miglyol (For example, 1 mg of GMFBI.1 drug:10 microliter of ethanol: 90 microliter of miglyol 812N).

Yet another aspect of the invention pertains to a method of treatment of DR comprising administration of the formulation comprising a therapeutically effective amount of GMFBI.1 represented by compound of formula (I), ethanol and miglyol812N at a ratio of 1 drug to 10 ethanol to 90 miglyol (For example, 1 mg of GMFBI.1 drug:10 microliter of ethanol: 90 microliter of miglyol812N).

### BRIEF DESCRIPTION OF FIGURES

The above-mentioned aspects, other features and advantages of the disclosure will be better understood and will become more apparent by referring to the exemplary embodiments of the disclosure, as illustrated in the accompanying drawings.

FIG. 1A: Image showing in silico illustration of affinity of 1H-Indazole-4-yl-methanol (GMFBI.1) towards carrier Miglyol812N. Hydrogen and hydrophobic interactions (dashed line) of GMFBI.1 with the solvent ethanol and Miglyol812N.

FIG. 1B: Image demonstrating the molecular electrostatic potential (MESP) map of carrier Miglyol812N featuring its hydrophobic pocket where GMFBI.1 (green) and ethanol (yellow) is bound.

FIG. 2A: Histogram showing the time-dependent plasma concentration of GMFBI.1 at different time points following subcutaneous (s.c.) administration (single dose) of GMFBI.1 (various amounts) in ethanol, Miglyol812N as a carrier in wild type (WT) C57BL/6 mice in comparison to s.c. administration of GMFBI.1 in saline and intraperitoneal (i.p.) administration of GMFBI.1 in saline 12 mg/kg (twice daily) positive control.

FIG. 2B: Histogram showing the time-dependent plasma concentration of GMFBI.1 following single dose p.o. administration of GMFBI.1 in ethanol, Miglyol812N as a carrier in wild type (WT) C57BL/6 mice in comparison to p.o. administration of GMFBI.1 in saline.

FIG. 2C: Histogram showing the Area under the curve (AUC: differential blood plasma concentration of GMFBI.1 as a function of time) created from pharmacokinetic data of GMFBI.1 administered vide s.c. and p.o. routes in comparison to established i.p. GMFBI.1 pharmacokinetic data. (n=3, mean±S.E.). Note that a single dose of 24 mg/Kg s.c once daily in Miglyol812N has a value similar to the 12 mg/Kg i.p. dose administered twice daily.

FIG. 3: Histogram depicting GMFBI.1 compound accumulation in WT C57BL/6 brain with respect to time for different s.c. doses of GMFBI.1 compound in ethanol Miglyol812N formulation compared to s.c. GMFBI.1 12 mg/kg in saline control. Note that GMFBI.1 given in ethanol, Miglyol812N formulation successfully penetrated the BBB compared to none seen following administration of GMFBI.1 in saline.

FIG. 4: Histogram depicting GMFBI.1 bioavailability in the brain following its administration by oral (p.o) route. BBB penetrability of GMFBI.1 following p.o. administration using Miglyol812N as a carrier in WT C57L/6 mice compared to saline as the vehicle.

FIG. 5: Sections of brain showing Amyloid β plaques in STZ-induced AD rat models. Thioflavin-S-stained brain sections showing the extent of plaques in healthy vs STZ induced AD vs GMFBI.1 in ethanol miglyol812N formulation treated STZ-induced AD rats following oral administration. Note the increased number of plaques in STZ-induced AD rats. Importantly, in orally administered GMFBI.1 treated animals, the number of plaques is reduced considerably indicating the effectiveness of the GMFBI.1 formulation in suppressing the induction of plaque formation.

FIG. 6: Immunoblot for GMF-β Ser83 phosphorylation in STZ-induced AD models. 25 μg of brain homogenate was subjected to SDS-PAGE and western blotting using anti-GMF-β phospho Ser83 antibody. Note the increased GMF-β Ser83 phosphorylation seen in STZ-induced AD rats which is decreased following treatment with GMFBI.1 in ethanol, miglyol812N formulation.

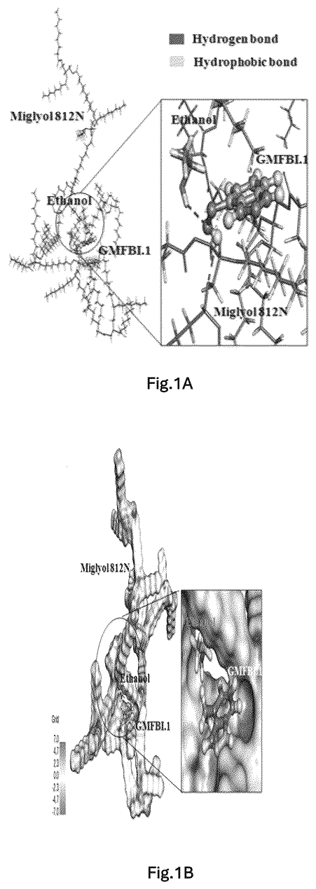

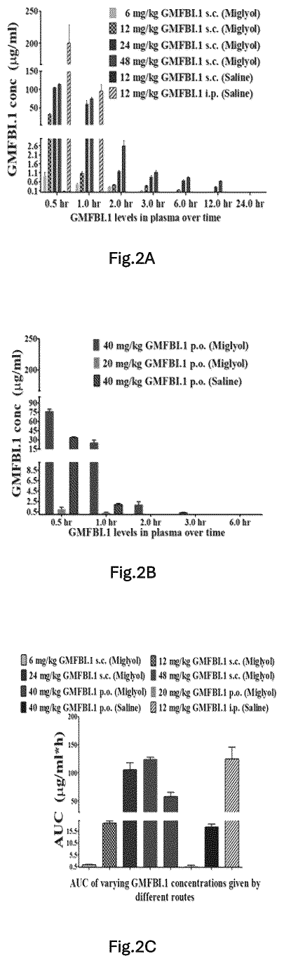

The figures depict exemplary embodiments of the disclosure for purposes of illustration only and not for limiting the scope intended to be covered.

### DETAILED DESCRIPTION OF INVENTION

While the invention has been disclosed with reference to certain embodiments, it will be understood by those skilled in the art that various changes may be made, and equivalents may be substituted without departing from the scope of the invention.

In addition, many modifications may be made to adapt to a particular situation or material to the teachings of the invention without departing from its scope.

Throughout the specification, the following terms take the meanings explicitly associated herein unless the context clearly dictates otherwise. The meaning of “a”, “an”, and “the” include plural references.

The meaning of “in” includes “in” and “on”. Referring to the drawings, like numbers indicate like parts throughout the views. Additionally, a reference to the singular includes a reference to the plural unless otherwise stated or inconsistent with the disclosure herein.

The terms “treat”, “treating”, and “treatment” refer to a method of alleviating or abrogating a disease and/or its attendant symptoms. The terms “prevent”, “preventing”, and “prevention” refer to a method of preventing the onset of a disease and/or its attendant symptoms or barring a subject from acquiring a disease. As used herein, “prevent”, “preventing”, and “prevention” also include delaying the onset of a disease and/or its attendant symptoms and reducing a subject's risk of acquiring a disease.

The phrase “therapeutically effective amount” or “pharmaceutically effective amount” means a compound, or a pharmaceutically acceptable salt thereof, sufficient to prevent the development of or to alleviate to some extent one or more of the symptoms of the condition or disorder being treated when administered alone or in conjunction with another therapeutic agent or treatment in a particular subject or subject population. For example, in a human or other mammal, a therapeutically effective amount can be determined experimentally in a laboratory or clinical setting or may be the amount required by the guidelines of the United States Food and Drug Administration, or equivalent foreign agency, for the particular disease and subject being treated. An aspect of the invention pertains to a pharmaceutical formulation comprising (1-H Indazol-4-yl)-methanol represented by Formula (I), or its pharmaceutically acceptable salt, ethanol and miglyol.

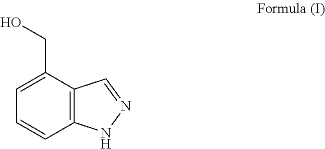 [SMILES: OCc1cccc2[nH]ncc12]

In an embodiment, the pharmaceutical formulation comprises (1-H Indazol-4-yl)-methanol represented by Formula (I), or its pharmaceutically acceptable salt, ethanol and miglyol at a ratio of 1:10:90. In other words, 1 part of drug, to 10 parts of ethanol to 90 parts of miglyol. (For example 1 mg of GMFBI.1 drug:10 microliter of ethanol:90 microliter of miglyol).

In various embodiments of the pharmaceutical formulation, the amount of (1-H Indazol-4-yl)-methanol or its pharmaceutically acceptable salt is present in the range of 1 mg to 500 mg preferably 5 mg to 100 mg, more preferably 10 mg to 40 mg per unit dose of said formulation.

In various embodiments, the pharmaceutical formulation further comprises additional agents such as diluents, excipients and/or pharmaceutically acceptable carriers to improve the absorption and retention characteristics of the formulation.

In various embodiments of the pharmaceutical formulation, the pharmaceutically acceptable carriers comprise glycerol, water, starch, mannitol; wherein the excipients comprise flavoring agents, preservatives, dispersing agents, anti-oxidants, buffers, bacteriostats, and coloring agents.

In various embodiments, the formulation has been formulated suitably so that it is suitable for administration either via oral (p.o.) route including buccal or sublingual route, rectal route or nasal route or topical route including transdermal route, peritoneal route, or vaginal route, or parenteral route including subcutaneous route, intramuscular route, intravenous route, or intradermal route.

In various embodiments, the pharmaceutical formulation is formulated as capsules or tablets; powders or granules; solutions or suspensions in aqueous or non-aqueous liquids; edible foams or whips; or oil-in-water liquid emulsions or water-in-oil emulsions, for administration via oral route.

In various embodiments, the pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions. Pharmaceutically acceptable carrier used herein, maybe a non-toxic, inert solid, semi-solid, or liquid filler, diluent, encapsulating material, or formulations auxiliary of any type. For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Oral fluids such as solutions of GMFBI.1-Miglyol 812N in Ethanol which include the drug, Miglyol 812N and Ethanol at a ratio of 1 drug, to 10 Ethanol to 90 Miglyol. (For example, 1 mg of GMFBI.1 drug:10 microliter of ethanol: 90 microliter of miglyol 812N) in the form of syrups, and elixirs can be prepared in dosage unit form so that a given quantity contains a predetermined amount of the compound.

In various embodiments, the pharmaceutical formulation is formulated as aqueous and non-aqueous sterile injection solutions, for administration via the parenteral route. Pharmaceutical formulations adapted for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

In various embodiments, the formulations of compound of formula (I) (GMFBI.1)-miglyol 812N in ethanol at a ratio of 1 drug to 10 ethanol to 90 miglyol (For example, 1 mg of GMFBI.1 drug:10 microliter of ethanol: 90 microliter of miglyol812N), may be presented in unit dose forms containing a predetermined amount of GMFBI.1 per unit dose. Such a unit dose may contain, for example, from 1 mg to 500 mg, preferably 5 mg to 100 mg, more preferably 10 mg to 40 mg of compound of formula (I) depending on the condition being treated, the severity of the condition, the time of administration, the route of administration, the rate of excretion of the compound employed, the duration of treatment, and the age, gender, weight, and condition of the patient as well as by the existence, nature and extent of any adverse side-effects in a subject.

In a specific embodiment, the formulation comprises a therapeutically effective amount of (1-H Indazol-4-yl)-methanol represented by Formula (I), or its pharmaceutically acceptable salt thereof along with, ethanol and Miglyol 812N suitable for the treatment of Alzheimer's disease, wherein the therapeutically effective amount ranges from 1 mg/Kg to 500 mg/Kg.

In a specific embodiment, the formulation comprises a therapeutically effective amount of (1-H Indazol-4-yl)-methanol represented by formula (I), or its pharmaceutically acceptable salt thereof along with, ethanol, miglyol812N suitable for the treatment of Stroke, wherein the therapeutically effective amount ranges from 1 mg/Kg to 500 mg/Kg.

In a specific embodiment, the formulation comprises a therapeutically effective amount of (1-H Indazol-4-yl)-methanol represented by formula (I), or a pharmaceutically acceptable salt thereof along with, ethanol and miglyol812N suitable for the treatment of Diabetic Retinopathy, wherein the therapeutically effective amount ranges from 1 mg/Kg to 500 mg/Kg.

In various embodiments, the compound of formula (I) is administered as a formulation in miglyol812N/ethanol in the range of 10-40 mg/kg body weight.

In various embodiments of the pharmaceutical formulation, the cell permeability of the pharmaceutical formulation is at least 500 nm/sec. In some embodiments, the oral absorption of the pharmaceutical formulation is at least 80%. In some embodiments, blood-brain barrier (BBB) permeability of the pharmaceutical formulation is in the range of −0.7 to −0.4. In some embodiments, the pharmaceutical formulation shows BBB permeability value in the range of −0.4 to −0.6, typically about −0.511.

In various embodiments of the formulation, the compound of formula (I) binds to hGMF-β with a binding affinity in the range of −6.5 to −5.5 kcal/mol. In some embodiments of the pharmaceutical formulation, the compound of formula (I) or its pharmaceutically acceptable salt binds to miglyol812 N and ethanol with a binding affinity of −5.3 kcal/mol.

In various embodiments, the pharmaceutical formulation is for use in the treatment of disease or disorder regulated by GMF-P, wherein the disease or disorder comprises Alzheimer's Disease, Stroke or Diabetic retinopathy.

In another aspect, the invention discloses a method of inhibiting hGMF-β phosphorylation wherein the method involves the administration of a formulation comprising a therapeutically effective amount of (1-H Indazol-4-yl)-methanol or its pharmaceutically acceptable salt, ethanol, and miglyol812N [at a ratio of 1 drug, to 10 ethanol to 90 miglyol (for example, 1 mg of GMFBI.1 drug:10 microliter of ethanol: 90 microliter of miglyol 812N)].

In another aspect, a method for treatment or prevention of a disease or disorder regulated by GMF-β is provided wherein the method involves the administration of a formulation comprising a therapeutically effective amount of (1-H Indazol-4-yl)-methanol or its pharmaceutically acceptable salt, ethanol and miglyol812N, at a ratio of 1:10:90 which is capable of penetrating the BBB.

In various embodiments of the method for the treatment or prevention of a disease or disorder regulated by GMF-P, the therapeutically effective amount of (1-H Indazol-4-yl)-methanol or its pharmaceutical salt is in the range of 1 mg/Kg to 500 mg/Kg.

Another aspect of the invention pertains to the method of preparation of said formulation comprising bringing into association the active ingredient with the carrier(s) or excipient(s).

Yet another aspect of the invention pertains to a method of treating Alzheimer's disease wherein the method comprises administration of formulation comprising a therapeutically effective amount of compound (1-H Indazol-4-yl)-methanol represented by formula (I), or its pharmaceutically acceptable salt thereof along with ethanol and miglyol812N; wherein the ratio of (1-H Indazol-4-yl)-methanol or its pharmaceutically acceptable salt, ethanol and miglyol812N in the formulation is 1:10:90.

In various embodiments of the method of treating Alzheimer's disease, the therapeutically effective amount of compound (1-H Indazol-4-yl)-methanol represented by formula (I), or its pharmaceutically acceptable salt ranges from 1 mg/Kg to 500 mg/Kg.

Yet another aspect of the invention pertains to a method of treating Stroke wherein the method comprises administration of formulation comprising a therapeutically effective amount of compound (1-H Indazol-4-yl)-methanol represented by formula (I), or its pharmaceutically acceptable salt thereof along with ethanol and miglyol812N wherein the ratio of (1-H Indazol-4-yl)-methanol or its pharmaceutically acceptable salt, ethanol and miglyol in the formulation is 1:10:90.

In various embodiments of the method of treating Stroke, the therapeutically effective amount of compound (1-H Indazol-4-yl)-methanol represented by formula (I), or its pharmaceutically acceptable salt thereof ranges from 1 mg/Kg to 500 mg/Kg.

Yet another aspect of the invention pertains to a method of treating Diabetic Retinopathy wherein the method comprises administration of formulation comprising a therapeutically effective amount of compound (1-H Indazol-4-yl)-methanol represented by formula (I), or its pharmaceutically acceptable salt along with ethanol and miglyol812N wherein the ratio of (1-H Indazol-4-yl)-methanol or its pharmaceutically acceptable salt, ethanol and miglyol in the formulation is 1:10:90.

In various embodiments of the method of treating Diabetic Retinopathy, the therapeutically effective amount of compound (1-H Indazol-4-yl)-methanol represented by Formula (I), or its pharmaceutically acceptable salt ranges from 1 mg/Kg to 500 mg/Kg.

In various embodiments, the formulation comprising compound (1-H Indazol-4-yl)-methanol represented by formula (I), or its pharmaceutically acceptable salt thereof along with miglyol812N and ethanol is non-toxic to human cells, typically human astrocytes, at concentrations of about 1 μg/ml to about 1 g/ml, typically about 0.01 mg/ml to about 1 mg/ml (or 0.065-6.5 mM).

In various embodiments, the method of treating Alzheimer's disease or stroke or diabetic retinopathy involves administration of the single dose or multiple doses of formulation daily.

In various embodiments, the method of treating Alzheimer's disease or stroke or diabetic retinopathy involves administration of the formulation having compound of formula(I) at an amount in the range of 1 mg/kg/day to 100 mg/kg/day, preferably in the range of 10 mg/kg/day to 15 mg/kg/day.

In various embodiments of methods of treating AD or stroke or DR, administration of the formulation decreases the progression of disease as observed by suppression of GMF-β Ser83 phosphorylation levels as well as suppression of proinflammatory cytokines expression.

In various embodiments of methods of treating AD or stroke or DR, administration of the formulation halts the progression of disease as observed by suppression of GMF-β Ser83 phosphorylation levels as well as suppression of proinflammatory cytokines expression.

In various embodiments of methods of treating AD or stroke or DR, administration of the composition increases the time of disease progression as observed by suppression of GMF-β Ser83 phosphorylation levels as well as suppression of proinflammatory cytokines expression.

In various animal models of AD, Stroke and DR, therapeutic efficacy of GMFBI.1 formulation in miglyol and ethanol has been monitored in terms of
(a) GMF-β Ser83 phosphorylation as a quantifiable end point showing suppression of inflammatory activity in animal models of AD, Stroke and DR administered either with GMFBI.1 formulation in miglyol812N and ethanol or with GMFBI.1 formulation in saline.
(b) Disease improvement manifested by recovery from inflammation measured in terms of reduced plaque pathology and tau phosphorylation and the likely cognitive improvement in the case of animal models of AD, administered either with GMFBI.1 formulation in miglyol812N and ethanol or with GMFBI.1 formulation in saline.
(c) Reduced inflammation as measured by the quantifiable amount of reduced GMF-β phosphorylation in the case of animal models of Stroke, administered either with GMFBI.1 formulation in miglyol812N and ethanol or with GMFBI.1 formulation in saline.
(d) Reduced neovascularization coupled with a quantifiable amount of reduced GMF-β phosphorylation in the case of animal models of DR, administered either with GMFBI.1 formulation in miglyol812N and ethanol or with GMFBI.1 formulation in saline.

As defined herein, a suitable amount or a therapeutically effective amount of the compound is assessed using techniques well known in the art such as by using animal models of AD/DR to obtain a concentration range and administration route. The information obtained from these models may be used to determine the route of administration and doses in humans.

Miglyol and Miglyol812N are interchangeably used to represent the same entity throughout the specification.

While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope.

### EXAMPLES

### Materials:

GMFBI.1 (1H-indazole-4yl-methanol) was obtained from Specs (Netherlands), ethanol, miglyol812N (medium-chain triglyceride, Glycerol Triester of caprylic and capric acid) were obtained from Sai Traders, India. Other chemicals such as isoflurane, streptozotocin, acetonitrile, ammonium bicarbonate, phosphate buffered saline, methanol and were obtained from Sigma-Aldrich, India. All animal experiments were performed in wild-type C57BL/6 female mice purchased from Tata Memorial Centre for Treatment Research and Education in Cancer (ACTREC, Mumbai) and Wild Type SD Rats were bred in-house animal facility as per the Institutional Animal Ethics Committee clearance AIMS Kochi (Ref. No. IAEC/2022/3/3).

### Preparation of Pharmaceutical Formulation:

GMFBI.1 (1H-indazole-4yl-methanol) is dissolved in 10% ethanol and 90% (v/v) miglyol 812N vehicle to obtain a formulation having GMFBI.1 (1H-indazole-4yl-methanol), ethanol and miglyol812N at a ratio of 1 part of drug:10 parts of ethanol: 90 parts of miglyol 812N. For example 1 mg of GMFBI.1 is dissolved in 10 microliter of ethanol and 90 microliter of miglyol 812N.

### Preparation of Animal Model of Alzheimer's Disease and Diabetic Retinopathy:

Diabetes is induced in adult wild-type SD rats by intraperitoneal injection of 40 mg/kg dose of Streptozotocin prepared in citrate buffer. The development of induced diabetes mellitus will be confirmed by examining the glucose level. Glucose levels will be measured prior to STZ administration and on a weekly basis after STZ administration. Glucose levels will be quantified using a commercial glucometer from the tail vein. A glucose level >250 mg/dL on day 3 confirms the development of diabetes mellitus. The diabetic rats with Alzheimer's pathology could be observed from three weeks of diabetic onset.

### Pharmacokinetic Studies:

### In-Vivo GMFBI.1 Bioavailability in Plasma Following its Subcutaneous (s.c.) and Oral (p.o) Administration in WT C57BL/6 Mice:

The pharmaceutical formulation of GMFBI.1 (1H-indazole-4yl-methanol) was prepared as described above. i.e. by dissolving GMFBI.1 in 10% ethanol and 90% (v/v) miglyol812N vehicle. Varying doses of GMFBI.1 were administered as a single bolus subcutaneously (s.c.), orally (p.o.) for time-dependent bio-distribution studies in wild-type (WT) C57BL/6 female mice (10-12 weeks old) (n=3 mice per group). Single-dose intraperitoneal (i.p) administration shown as control is an alternative route of delivery. Eventually, WT mice were sacrificed at 0.5, 1, 2, 3, 6, 12 and 24 hrs (for higher GMFBI.1 doses) post GMFBI.1 s.c. injection and 0.5, 1, 2, 3, and 6 hrs respectively after p.o. administration by exsanguination via cardiac puncture under isoflurane anesthesia.

Quantification of GMFBI.1 present in tissues was carried out using reverse-phase high-performance liquid chromatography (RP-HPLC). Approximately 1 ml of blood was collected from mice into heparinized vacutainers (BD vacutainers, USA) for separation of plasma. Key organs brain, heart, liver, lung, kidney, spleen, stomach, and intestine were also collected, immediately rinsed in phosphate-buffered saline, snap frozen in liquid nitrogen, and stored at −80° C. until further analysis. Concentration analysis of GMFBI.1 in plasma and tissues of s.c. and p.o. administered C57BL/6 mice was carried out using RP-HPLC. Briefly, GMFBI.1 was extracted from plasma and tissue homogenate by adding 3:1 ratio of acetonitrile and water to 100 μl of sample for the proteins to be precipitated by keeping overnight at −20° C. The next day, samples were vortexed for 10 secs and centrifuged at 13000 rpm for 10 min at 4° C. The supernatant obtained was vaporized to dryness using a rotary evaporator (Labconco, Switzerland). Samples were then re-suspended in the mobile phase (75:25, Ammonium bicarbonate:acetonitrile (v/v)) and subjected to HPLC analysis using a LC-20AD-prominence HPLC (Shimadzu, Japan) equipment. C18 column (Qualisil BDS C18 chromatography column, 5 μm, 4.6×250 nm, USA) was used for resolving GMFBI.1 peak. Retention time for the compound was found to be 4.0±0.9 min at a fixed flow rate of 1 ml/min. The amount of GMFBI.1 in plasma and concerned organs was quantified from the standard plot. Pharmacokinetic parameters for various groups (both s.c. and p.o.) were derived using PKSolver software using a non-compartmental analysis method.

### In-Vivo GMFBI.1 Bioavailability in the Brain Following its Administration by Subcutaneous (s.c.) Route:

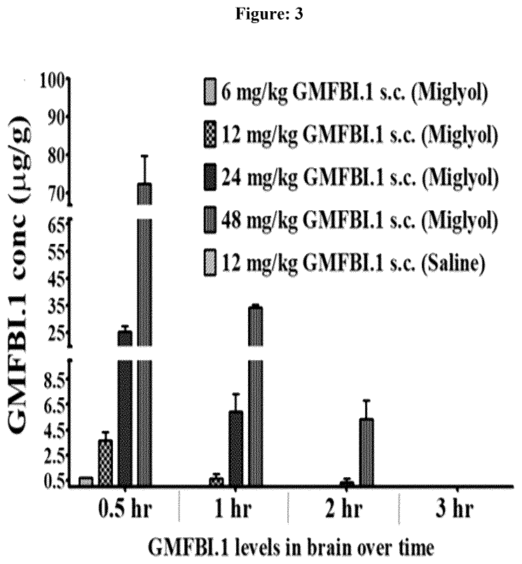

The pharmaceutical formulation of GMFBI.1 (1H-indazole-4yl-methanol) was prepared as described above. i.e. by dissolving GMFBI.1 in 10% ethanol and 90% (v/v) miglyol812N vehicle. Varying doses of GMFBI.1 were given as a single bolus subcutaneously (s.c.) for time dependent bio-distribution studies in Wild Type (WT) C57BL/6 female mice (10-12 weeks old) (n=3 mice per group). Eventually, WT mice were sacrificed at 0.5 hr, 1 hr, 2 hrs, 3 hrs, post GMFBI.1 s.c. administration by exsanguination via cardiac puncture under isoflurane anesthesia. GMFBI.1 compound accumulation in WT C57BL/6 brain with respect to time for different s.c. doses in miglyol compared to s.c. GMFBI.1 12 mg/kg saline control. Results are shown in FIG. 3. Quantification of GMFBI.1 present in the brain tissue was carried out using reverse-phase high-performance liquid chromatography. It was observed that GMFBI.1 administered via ethanol, miglyol812N formulation showed higher amounts of GMFBI.1 in brain tissues demonstrating successful penetration of GMFBI.1 through the BBB. Whereas no GMFBI.1 could be detected in the brain of mice administered with a formulation of GMFBI.1 in saline demonstrating its inability to penetrate BBB.

### GMFBI.1 Bioavailability in the Brain Following its Administration by Oral (p.o) Route:

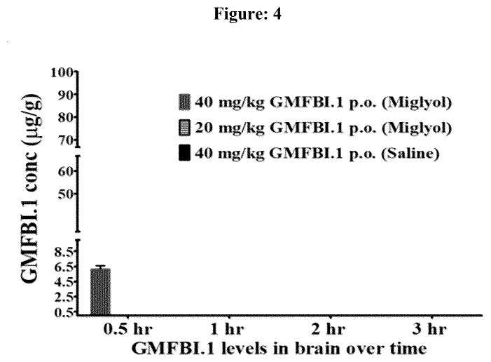

The pharmaceutical formulation of GMFBI.1 (1H-indazole-4yl-methanol) was prepared as described above. i.e. by dissolving GMFBI.1 in 10% ethanol and 90% (v/v) miglyol812N vehicle. Varying doses of GMFBI.1 were given as a single bolus orally (p.o) for time-dependent bio-distribution studies in Wild Type (WT) C57BL/6 female mice (10-12 weeks old; n=3 mice per group). Eventually, WT mice were sacrificed at 0.5, 1, 2, 3 h post GMFBI.1 p.o. administration by exsanguination via cardiac puncture under isoflurane anesthesia. Results are shown in FIG. 4. Quantification of GMFBI.1 present in the brain tissue was carried out using reverse-phase high-performance liquid chromatography. GMFBI.1 penetrated the BBB following p.o. administration of the formulation of GMFBI.1 in ethanol and miglyol812N, whereas GMFBI.1 when administered using saline as a carrier could not penetrate the BBB and hence could not be detected.

### Induction of Alzheimer's and Diabetic Retinopathy Rat Model Using Streptozotocin.

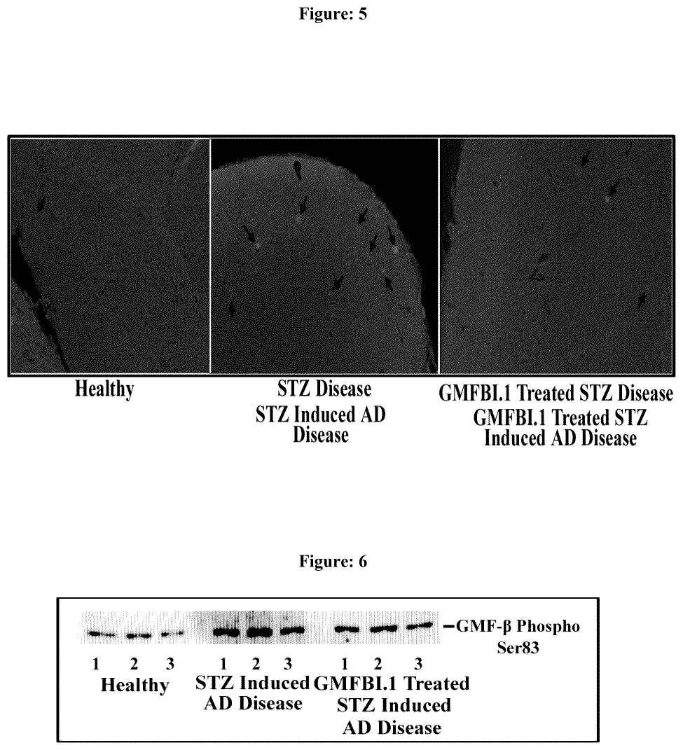

Suppression of plaque formation following treatment with GMFBI.1: Alzheimer's pathology was created in streptozotocin-induced SD rats as explained above. Diabetes is induced in rats by intraperitoneal injection of streptozotocin 40 mg/kg prepared in citrate buffer. On the fourth day the rats were orally administered with GMFBI.1 in ethanol and Miglyol812N formulation for the next 5 weeks. The brain and the eye were harvested. One half of the brain was frozen for performing GMF-β Ser83 phosphorylation analysis and the other portion was fixed in paraformaldehyde (4%) and 5 μm sections were taken. Brain sections were stained with Thioflavin-S, a stain for detecting the plaques. The extent of plaque formation was assessed by Thioflavin S staining in healthy, GMFBI.1 treated and untreated animals (FIG. 5). Thioflavin-S-stained brain sections showed an increased number of plaques in STZ-induced AD rats, whereas there were no plaques in healthy rats and very less plaques in GMFBI.1 treated STZ-induced AD rats. Importantly, it was found that oral administration of GMFBI.1 in ethanol and Miglyol812N formulation considerably reduced the number of plaques indicating the effectiveness of GMFBI.1 formulation in suppressing the induction of plaque formation.

### Suppression of GMF-β Ser83 Phosphorylation by GMFBI.1 Treatment in STZ Induced AD Rat Brains.

Western blot analysis was performed using the brain homogenate obtained from healthy rats, STZ-induced AD rats, and STZ-induced AD rats treated with GMFBI.1 formulation (orally administered with GMFBI.1 in ethanol and Miglyol812N formulation for 5 weeks). Western blot showed an increased Ser83 phosphorylation of GMF-β in the brain tissues of STZ-induced AD rats when compared to the brain tissues obtained from healthy rats (FIG. 6). Further, it was also found that treatment with GMFBI.1 formulation resulted in a drastic reduction in GMF-β Ser83 phosphorylation indicating that activity of GMF-β activity is suppressed by GMFBI.1 formulation administration. Neuroinflammation is one of the key events that augments the plaque formation and the eventual neuronal damage and cognitive defects seen in AD. Suppression of GMF-β activity by GMFBI.1 formulation administration resulted in the suppression of proinflammatory response which correlated with reduced plaque formation reduced neuronal damage and reduced cognitive defects. Thus, it is evident from the data on the animal model of AD that GMFBI.1 formulation in Miglyol812N is capable of inhibiting the incidence of a number of plaques compared to untreated animals. This formulation was developed mainly to address the issues of poor bioavailability of the GMFBI.1 compound when administered in saline is very poor due to increased clearance and its inability to cross the blood-brain barrier (FIG. 4). However, when GMFBI.1 is administered in ethanol Miglyol812N formulation, GMFBI.1 efficiently crossed the BBB even in WT animals when administered orally (FIG. 4).

The oral administration route is chosen for administering the GMFBI.1 formulation in Streptozotocin (STZ) induced animal models of diabetes since the involvement of the gut-brain axis is well documented in neurodegenerative diseases such as AD and multiple sclerosis, due to the close interaction between the enteric nervous system and central nervous system and also because of the correlation observed between bioavailability of the drug in the gut and the complete recovery from said diseases. The advantage of employing Streptozotocin (STZ) induced animal models of diabetes is that in the same animal brain, it induces AP plaques (the AD model) and in the eye it induces diabetic retinopathy (DR). From the results shown in FIGS. 5 and 6, it is clear that administration of the drug GMFBI.1 in the Miglyol812N formulation, AP plaque formation and GMF-β phosphorylation in the brain could be suppressed. Since DR also results in the eye of the same animal following STZ administration, similar suppression of GMF-β phosphorylation happens in the eye or retina as seen in the brain (AD model) thereby preventing the inflammation, as it is well documented by several studies that GMF-β is upregulated in the retinal cells (Caiying Liu et al, Juan Wang et al, Liu J et al).

### Advantages

Formulation comprising (1H-indazol-4-yl) methanol or its pharmaceutically acceptable salt, Ethanol and Miglyol 812N, at a ratio of 1:10:90 helps in penetrating the BBB and retention of the compound in the system thereby increasing its bioavailability. The compound (1H-indazol-4-yl) methanol of the formulation specifically binds to hGMF-β and blocks the Ser83 sites specified residues resulting in the blockage of phosphorylation of hGMF-β leading to suppression of pro-inflammatory response mediated by astrocytes and microglia related to inflammation including Alzheimer's, Stroke and Diabetic Retinopathy. The disclosed formulation is useful for treating these diseases.

### REFERENCES

1. Caiying Liu, Wan Sun, Tong Zhu, Si Shi, Jieping Zhang, Juan Wang, Furong Gao, Qingjian Ou, Caixia Jin, Jiao Li, Jing-Ying Xu, Jingfa Zhang, Haibin Tian, Guo-Tong Xu, Lixia Lu, (2022). GMF-β induces ferroptosis in early diabetic retinopathy by impairing chaperone-mediated autophagic degradation of ACSL4, Redox Biol. 52, 102292, https://doi.org/10.1016/j.redox.2022.102292
2. Juan Wang; Maihemuti Awuti; Xiaoman Jiang; Jieping Zhang; Yali Lyu; Lixia Lu; Guo-Tong Xu (2018). Effect of Glia Maturation Factor beta (GMFB) on Retinal Pigment Epithelium in Diabetic Retinopathy. Investigative Ophthalmology & Visual Science, Vol. 59, 3979.
3. Liu J, Hou Y, Lin L, Yu N, Zhang Y (2021) MicroRNA-5195-3p alleviates high glucose-induced injury in human ARPE-19 cells by targeting GMFB. PLoS ONE 16(11): e0260071.

1. A pharmaceutical formulation comprising (1-H Indazol-4-yl)-methanol represented by formula (I), 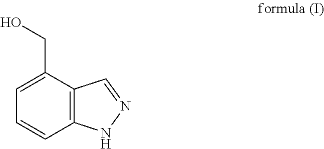 [SMILES: OCc1cccc2[nH]ncc12]
or its pharmaceutically acceptable salt, ethanol, and miglyol.

2. The pharmaceutical formulation as claimed in claim 1, wherein the ratio of (1-H Indazol-4-yl)-methanol or its pharmaceutically acceptable salt, ethanol, and miglyol is 1:10:90.

3. The pharmaceutical formulation as claimed in claim 1, wherein (1-H Indazol-4-yl)-methanol or its pharmaceutically acceptable salt is present in the range of 1 mg to 500 mg, preferably 5 mg to 100 mg, more preferably 10 mg to 40 mg per unit dose of said formulation.

4. The pharmaceutical formulation as claimed in claim 1, further comprises diluents, excipients, and/or pharmaceutically acceptable carriers.

5. The pharmaceutical formulation as claimed in claim 4, wherein the pharmaceutically acceptable carriers comprise glycerol, water, starch, mannitol; wherein the excipients comprise flavoring agents, preservatives, dispersing agents, anti-oxidants, buffers, bacteriostats, and coloring agents.

6. The pharmaceutical formulation as claimed in claim 1, for administration via oral route including buccal route or sublingual route, rectal route or nasal route or topical route including transdermal route, peritoneal route, or vaginal route, or parenteral route including subcutaneous route, intramuscular route, intravenous route, or intradermal route.

7. The pharmaceutical formulation as claimed in claim 1, wherein the formulation is formulated as capsules or tablets; powders or granules; solutions or suspensions in aqueous or non-aqueous liquids; edible foams or whips; or oil-in-water liquid emulsions or water-in-oil emulsions, for administration via oral route.

8. The pharmaceutical formulation as claimed in claim 1, wherein the formulation is formulated as aqueous and non-aqueous sterile injection solutions, for administration via parenteral route.

9. The pharmaceutical formulation as claimed in claim 1, wherein cell permeability of the formulation is at least 500 nm/sec; oral absorption of the formulation is at least 80%; blood-brain barrier (BBB) permeability of the formulation is in the range of −0.7 to −0.4.

10. The pharmaceutical formulation as claimed in claim 1, wherein (1-H Indazol-4-yl)-methanol or its pharmaceutically acceptable salt binds to miglyol and ethanol with a binding affinity of −5.3 kcal/mol.

11. The pharmaceutical formulation as claimed in claim 1, wherein the formulation is non-toxic to human cells, at concentrations of about 1 μg/ml to about 1 g/ml, preferably about 0.01 mg/ml to about 1 mg/ml.

12. The pharmaceutical formulation as claimed in claim 1, for use in the treatment of disease or disorder regulated by GMF-β.

13. The pharmaceutical formulation as claimed in claim 12, wherein the disease or disorder comprises Alzheimer's, Stroke or Diabetic retinopathy.

14. A method for treatment of a disease or disorder regulated by GMF-β wherein the method comprises administration of a formulation comprising a therapeutically effective amount of (1-H Indazol-4-yl)-methanol or its pharmaceutically acceptable salt, ethanol and miglyol; wherein the ratio of (1-H Indazol-4-yl)-methanol or its pharmaceutically acceptable salt, ethanol and miglyol in the formulation is 1:10:90; wherein the formulation is capable of penetrating the BBB.

15. The method as claimed in claim 14, wherein the therapeutically effective amount of (1-H Indazol-4-yl)-methanol or its pharmaceutically acceptable salt is in the range of 1 mg/Kg to 500 mg/Kg.

16. A method of treatment of Alzheimer's disease, Stroke or Diabetic retinopathy, wherein the method comprises administration of formulation comprising a therapeutically effective amount of (1-H Indazol-4-yl)-methanol or its pharmaceutically acceptable salt, ethanol and miglyol; wherein the ratio of (1-H Indazol-4-yl)-methanol or its pharmaceutically acceptable salt, ethanol and miglyol in the formulation is 1:10:90.

17. The method as claimed in claim 16, wherein the therapeutically effective amount of (1-H Indazol-4-yl)-methanol or its pharmaceutically acceptable salt is in the range of 1 mg/Kg to 500 mg/Kg.

18. The method as claimed in claim 16, wherein the method of treating Alzheimer's disease or stroke or diabetic retinopathy involves the administration of a single dose or multiple doses of formulation daily.

19. The method as claimed in claim 16, wherein the amount of (1-H Indazol-4-yl)-methanol or its pharmaceutically acceptable salt is administered through the formulation at an amount in the range of 1 mg/kg/day to 100 mg/kg/day, preferably in the range of 10 mg/kg/day to 15 mg/kg/day.
